(19) United States
(12) Patent Application Publication
(10) Pub. No.: US 2019/0000381 A1
(43) Pub. Date: Jan. 3, 2019

(54) METHOD FOR DETERMINING AUTOMATICALLY THE DICHOTOMY INDEX I<O OF AN INDIVIDUAL

(71) Applicants: UNIVERSITE DE TECHNOLOGIE DE TROYES, Troyes Cedex (FR); ALTRAN TECHNOLOGIES, Neuilly-sur-Seine (FR)

(72) Inventors: Aly CHKEIR, Troyes (FR); Jacques DUCHENE, La Riviere De Corps (FR); David James HEWSON, Wheathampstead (GB); Jacques Jean BEAU, Villabé (FR); Francis Albert LEVI, Villejuif (FR)

(21) Appl. No.: 16/063,619

(22) PCT Filed: Dec. 16, 2016
(86) PCT No.: PCT/EP2016/081576

§ 371 Date: Jun. 18, 2018

(30) Foreign Application Priority Data
Dec. 16, 2015 (FR) 1562557

(51) Int. Cl. A61B 5/00; A61B 5/01; A61B 5/11

(52) U.S. Cl. CPC A61B 5/4857; A61B 5/01; A61B 5/1118; A61B 2562/0271; A61B 5/6823; A61B 2562/0219; A61B 5/4809

(57) ABSTRACT

The object of the present invention is to propose a method for automatically determining the dichotomy index I<O of an individual, by proposing a means for automatically and specifically detecting the activity and rest states on the basis of the dichotomy index applicable to all individuals including persons having very low activity such as elderly persons, or hospitalized persons without using the thresholding methods applied by prior algorithms.

### TECHNICAL FIELD OF THE INVENTION

The invention relates to the field of methods for determining the dichotomy index of an individual, i.e. the index identifying the regularity of the alternation of day activity and night rest as well as their amplitude over 24H giving the possibility of providing a measurement of the circadian rhythm.

### STATE OF THE PRIOR ART

Disruptions of the circadian system which may be caused by working in shifts significantly increase the risk of cancer, notably breast, colon and prostate cancer. Also, perturbation of the activity—rest circadian rhythms, measured by actimetry, represents a negative prognostic factor in terms of survival in patients affected with metastatic colon, breast, kidney, ovary or lung cancer, independently of known prognostic factors.

Actimetry consists in a non-invasive technique for measuring the activity/rest cycle and of the sleep-wake rhythm: the circadian rhythm. To do this, an actimeter is used, appearing as a casing provided with at least one accelerometer and which records the successive zero-crossings of the acceleration.

This type of device coupled with a server allowing analysis of the collected data gives the possibility of reliably estimating periods of activity and of sleep, well correlated with those detected by polysomnography, with however greater uncertainty for falling asleep than for awaking.

Although the actimeter is considered as the “gold standard” for measuring activity/rest, it is not ideal for individuals admitted to hospital with a severe pathology and/or which have very reduced mobility, and therefore low physical activity. This is notably due to the fact that the activity levels are considerably lower than those of healthy individuals which makes it extremely difficult to make a distinction between the rest phase and the wakefulness phase, in particular for falling asleep.

The usual methods for detecting activity in this type of individual, adapt the detection thresholds by using parametric or non-parametric algorithms based on smoothing, logical combinations or approaches based on artificial neurone networks as disclosed in Tilmanne, J. “algorithms for sleep-wake identification using actigraphy: a comparative study and new results” Journal of Sleep Research 2009, 18, (1) pp 85-98.

In particular, these are neural networks which give the best result. However, in spite of these algorithmic progress, a percentage of errors persists at the detection of the breach between rest and awareness for hospitalized individuals.

### DISCUSSION OF THE INVENTION

The present invention therefore has the purpose of proposing a method for automatically determining the dichotomy index I<O of an individual, giving the possibility of at least overcoming a portion of the drawbacks of the prior art, by proposing a means for automatically and specifically detecting the activity and rest states on the basis of the dichotomy index which may be applied to all individuals, including persons having very low activity such as elderly persons, or hospitalized persons without using the thresholding methods applied by the prior algorithms.

For this purpose, the invention relates to a method for automatically determining the dichotomy index I<O of an individual, from at least data from a system comprising:
a portable module composed of at least:
   a temperature sensor generating a signal of data representative of the body temperature of the individual,
   an accelerometer simultaneously generating two data signals, a first “ZCM” signal representative of the activity of the individual over time and a second “positionX” signal representative of the tilt of the individual with respect to the ascending vertical over time,
a computer server provided with at least one processor, one memory, and a display means and configured for receiving the data signals from the portable sensor, and for carrying out said method,
   characterized in that the method comprises at least the following steps:
A. receiving and recording in the memory, by the processor, of the various data of the temperature, ZCM and positionX signals of the portable module,
B. suppressing from the memory by the processor of the non-utilizable data from the module when the latter is not worn by the individual,
C. identifying and resetting to zero by the processor of the aberrant data from the ZCM and positionX signals of the accelerometer and of their recordings in the memory,
D. transforming the values of the positionX signal of the accelerometer by the processor into binary values (0 or 1) corresponding to an alert state (0) or lying-down state (1) of the individual and their recordings in the memory,
E. transforming the values of the ZCM signal of the accelerometer by the processor into binary values (0 or 1) corresponding to an alert or lying-down state of the individual and their recordings in the memory,
F. comparing the binary values of the ZCM and positionX signals of the accelerometer by the processor and identifying at least one lying-down state of the individual constituted by an uninterrupted succession of a binary value identical for the signals of the accelerometer over a time period greater than or equal to 180 minutes,
G. calculating the dichotomy index and its display on the display means, by the processor by following the following equation:

$$I < 0 = ( 1 - \frac{{NB}_{C}}{{NB}_{L}} ) \times 100$$

with:
NB_{C}, the number of values of the first ZCM signal representative of the activity of the individual versus time, located in the lying-down state identified during step E, which are greater than the median of the values of the first ZCM signal representative of the activity of the individual versus time located outside the lying-down state identified during step E.
NB_{L}, the number of values of the first signal relative to the activity (ZCM) of the individual, located outside the lying-down state identified during step E.

The dichotomy index I<O, represents the percentage of activity, per minute during the rest period (usually at night), which is less than the median of activity away from the bed (usually during daytime). It is a reliable indicator of the activity-rest circadian rhythm. It is calculated from the measurement of the number of accelerations per minute and from the position of the patient by means of a thorax actimeter worn for at least three consecutive days. This is a non-invasive measurement, generally well accepted by the patients.

“ZCM” or the Zero Crossing Mode corresponds to the number of times that the signal passes through 0 for each time period.

“positionX” corresponds to the value of the angle formed by the accelerometer relatively to the ascending vertical when the latter is worn on the chest of the individual.

According to a particularity, the data signals comprise values taken at a regular interval over a time period of 24 hours.

According to another particularity, when the number of values of the data signals for the temperature sensor and the accelerometer are different, the processor carries out a normalization step on the data recorded in the memory prior to step B, by using a cubic spline polynomial interpolation so that each value from the temperature signal is assigned to a value of the positionX and ZCM signals, and said values are recorded in the memory of the system.

According to another particularity, during step B, the processor suppresses from the memory all the values of temperature data strictly less than 30° C. and the values of the positionX and ZCM signals which are associated in time with the temperature value strictly less than 30° C.

According to another particularity, during step C, the processor independently applies on the whole of the positionX and ZCM data the following sub-steps:
a) Selecting with the processor in the memory a first block of consecutive values,
b) Sorting in an increasing order the values of the block,
c) Suppressing value repetitions of the block, in order to obtain a unique data set,
d) Calculating the median of the unique data set,
e) Resetting to zero in the memory the values of the positionX and ZCM data signals when their value is greater than the median,
f) Repeating steps a) to e) on the next block of consecutive values.

According to another particularity, the blocks of values are constituted by 31 values corresponding to a time period of 31 minutes of the signals, the median is calculated over the first 10 data of the unique data set and the reset to zero is accomplished by sub-sets of 5 data corresponding to 5 consecutive minutes, if there are less than 2 values greater than the median, then the processor resets to zero in the memory the corresponding sub-set.

According to another particularity, during step D, on the whole of the positionX signal, the processor
determines and records in the memory, the minimum median by carrying out the following sub-steps:
sorts the values in an increasing order,
suppresses the value repetitions, in order to obtain a unique data set, and
calculates the minimum median over the first 31 values of the unique data set,
determines and records in the memory, the maximum median by carrying out the following sub-steps:
sorts the values in decreasing order,
suppresses the value repetitions, in order to obtain a unique data set, and
calculates the maximum median over the first 31 values of the unique data set,
determines and records in the memory, the threshold median corresponding to the average of the minimum and maximum medians,
transforms and records, in the memory, the signal of positionX data by replacing with 0 the values strictly less than the threshold median, and with 1 the values greater than or equal to the latter in order to obtain a positionX_binary data signal,
smoothes out the values recorded in the memory of the positionX_binary data signal, by the processor when a set of data delimited by non-identical values corresponds to a time period of less than 1 h30, the values of said data set are then reversed, from 0 to 1 or from 1 to 0.

According to another particularity, during step E, on the whole of the ZCM signal, the processor:
normalizes the ZCM values by dividing each ZCM value by the maximum value of the signal in order to obtain a new ZCM_time signal, the values of which vary between 0 and 1.
applies to the ZCM_time signal a variance filter over 10 points and records the signal in the memory
calculates the accumulated quadratic average from the ZCM_time signal,
searches for a plateau on the data of the accumulated quadratic average by carrying out with the processor the following sub-steps:
   multiplication of each value of the quadratic average by a factor of 10⁴ and calculation of the rounded to the nearest integer
   reading with a pitch of 2 units a window corresponding to 200 units,
   determining the number of points in each window, if the number of found points corresponds to a time period greater than 180 min, the processor increments a “plateau” counter and retains in memory the time index of the first and of the last point of the plateau
   transforming and recording in the memory, the ZCM data signal by replacing the values located between the time indexes of the plateau with 1 and the other ones with 0 in order to obtain a ZCM_binary data signal.

According to another particularity, during step F, the processor carries out a comparison of the ZCM_binary and positionX_binary data signals in order to determine the time indexes of the plateau for which the initial and final data values correspond to 1, the lying-down state.

According to another particularity, during step G, the processor does not take into account for calculating the dichotomy index, the data corresponding to a time period of one hour before and of one hour after the beginning of the plateau and one hour before and one hour after the end of the plateau.

### SHORT DESCRIPTION OF THE FIGURES

Other features, details and advantages of the invention will become apparent upon reading the description which follows with reference to the appended figures:

FIG. 1, illustrates an example of values of the positionX signal (step A),

FIG. 2, illustrates the same example of values of the positionX signal after suppressing the non-utilizable data due to the fact that the sensor is not worn (step B),

FIG. 3, illustrates the same example of values of the positionX signal after applying the filter for the aberrant data (step C),

FIG. 4, illustrates an example of a positionX binary value corresponding to the same example of values of the positionX signal before smoothing out (step D),

FIG. 5, illustrates the same example of positionX binary values corresponding to the same example of values of the positionX signal after smoothing (step D),

FIG. 6, illustrates an example of a ZCM_time value of the ZCM signal before applying the variance filter (step E),

FIG. 7, illustrates the same example of a ZCM_time value of the ZCM signal after applying the variance filter (step E),

FIG. 8, illustrates the same example of a ZCM_time value of the ZCM signal after applying the variance filter with the accumulated quadratic average (step E),

FIG. 9 illustrates, the ZCM_binary values after transformation of the ZCM signal (step E),

FIG. 10 illustrates the values required for calculating the dichotomy index

FIG. 11 illustrates the readout window allowing determination of the existence of a plateau, during step E.

### DETAILED DESCRIPTION OF DIFFERENT EMBODIMENTS OF THE INVENTION

A non-limiting example of an embodiment of the method for automatically determining the dichotomy index I<O of an individual according to the invention is now described. It uses data from a portable module composed of at least:
a temperature sensor generating a data signal representative of the body temperature of the individual,
an accelerometer simultaneously generating two data signals, a first ZCM signal, representative of the activity of the individual over time and a second positionX signal representative of the tilt of the individual with respect to the ascending vertical over time.

As an example, the accelerometer used may be the “ADXL345” accelerometer capable of generating a readout of the activity (or “ZCM” Zero Crossing Mode ZCM (zero crossing mode) which corresponds to the number of times during which the signal crosses 0 for each time period) and of the tilt (or “positionX”) every minute. The temperature sensor is preferably a sensor of the infrared type configured for measuring the body temperature every 5 minutes.

The portable module may be connected through a wireless communication circuit with a collector module for example through a Bluetooth, WIFI, or GPRS connection. Said collector module communicates the signals to the server. This communication may be GPRS, Bluetooth, WIFI, LIFI, infrared, radio or wired by means of a communication circuit of a computing resources, for example a server including a processing unit, for example a processor, a memory and a program using the data temporarily stored in the memory of the module and issued to the memory of the server through the communication circuit.

In some embodiment, the portable module does not require to be connected to the server. Indeed, the portable module can include the computing resources as a processing unit, for example a processor, a memory and a program using the data from the sensor and accelerometer, temporarily stored in the memory of the module, to perform the method for automatically determining the dichotomy index I<O of an individual. In other words, the portable module and the function of the server can be reunited in one portable element.

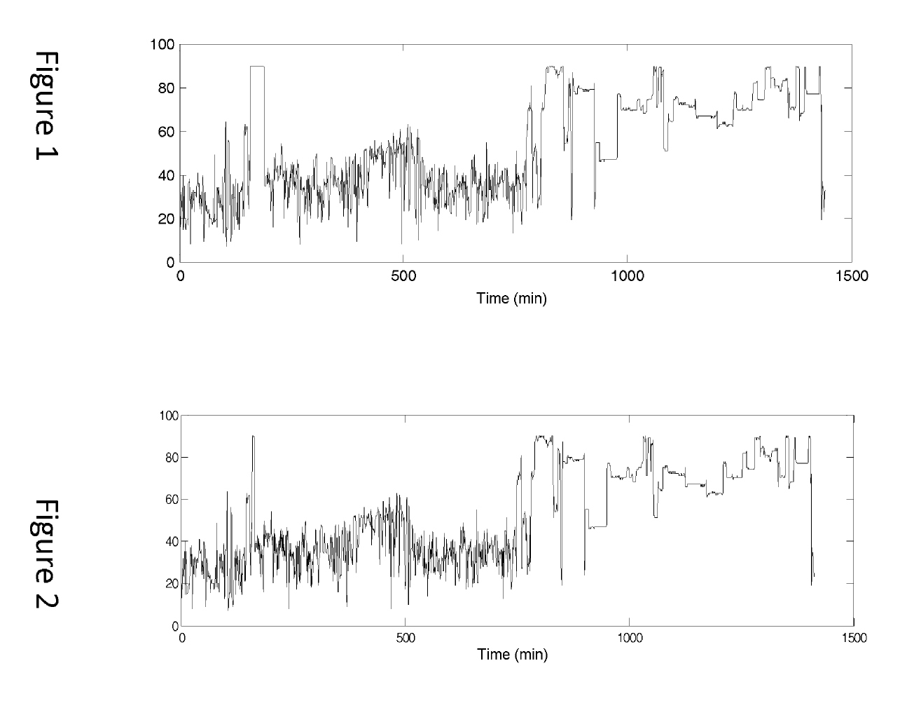

Thus, every 24 hours, the server receives the measurements corresponding to 3 data signals, a first signal corresponding to a succession of representative values of the body temperature of the individual for example taken every 5 minutes over a 24-hour period, (a higher frequency may of course be used but is not necessarily relevant given that the variation of the body temperature is slow), a second “ZCM” signal corresponding to a succession of values representative of the activity of the individual for example taken every minute over a period of 24 hours and a third “positionX” signal (FIG. 1), corresponding to a succession of values representative of the tilt of the individual with respect to the ascending vertical, these values being taken for example every minute over a period of 24 hours.

A “24-hours period” is understood to mean: a period of 24 hours which can include a corrective factor (which can be added to, or subtracted from, this time period) of the order of a few minutes to a maximum of the order of one hour. This correction factor may for example be calculated on the basis of previous measurements for an individual, in order to determine the duration of these cycles, for example over one or more past cycles. This factor can also be determined on the basis of statistical estimates of the duration of future cycles of the individual, for example on the basis of past measures and/or various predictive factors.

In order to obtain as many temperature values as there are “positionX” or “ZCM” values, the server carries out a normalization step on the temperature data recorded within the memory, by using a cubic spline polynomial interpolation so that every temperature readout gives 5 values in order to obtain a temperature value for each “positionX” and “ZCM” value versus time.

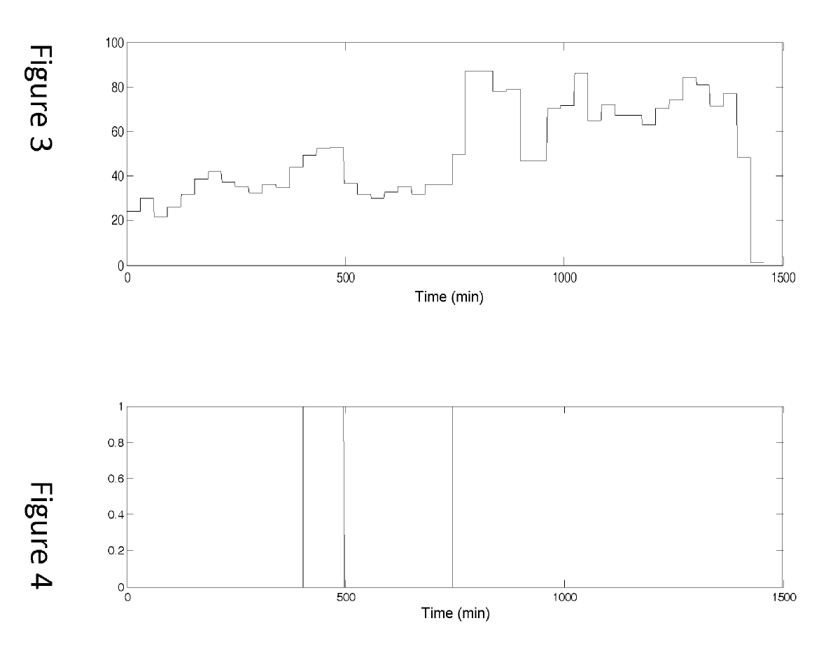

Subsequently, a step for suppressing the data generated by the portable module when it is not worn is carried out by the server. For this purpose, the server includes a program allowing during its execution, identification of the temperature values of less than 30° C. as well as their time index, i.e. the time reference for the taking of the measurement. This algorithm suppresses from the memory the temperature values of less than 30° C. as well as the “ZCM” and “positionX” values having the same time index (FIG. 2).

A step for correcting the values from the “positionX” tilt signal may be carried out in order to not be found with negative angular values when the portable module is worn upside down. For this, when the “positionX” value is greater than 90° than the program executed by the server carries out the following transformation by subtracting from 180 the “positionX” value: 180−“positionX” value.

Subsequently, the server carries out an identification and zero reset step by the processor of the aberrant data from the “ZCM” and “positionX” signals. To do this, the processor independently carries out the following sub-steps on the whole of the “positionX” and “ZCM” data:
a) Selecting with the processor, in the memory of a first X block (for example, 31) consecutive values corresponding to a duration X (for example 31 minutes) of significant measurements,
b) Sorting in an increasing order the values of the block,
c) Suppressing value repetitions of the block, in order to obtain a unique data set,
d) Calculating the median of the first values of the unique data set,
e) Reading the block of X (31) values per Y sub-block (for example, 5) data corresponding to Y (respectively 5) measurement minutes, and resetting to zero in the memory of the Y sub-block (for example 5) data when there are less than Z<V (for example 2) values greater than the median,
f) Repeating steps a) to e) on the next block of consecutive values. (FIG. 3).

Following the step for removing the aberrant values “ZCM” and “positionX”, the server transforms the values of the “positionX” and “ZCM” signals into binary values 0 or 1. The binary value 1 corresponds to a rest state and the binary value 0 to a state of activity.

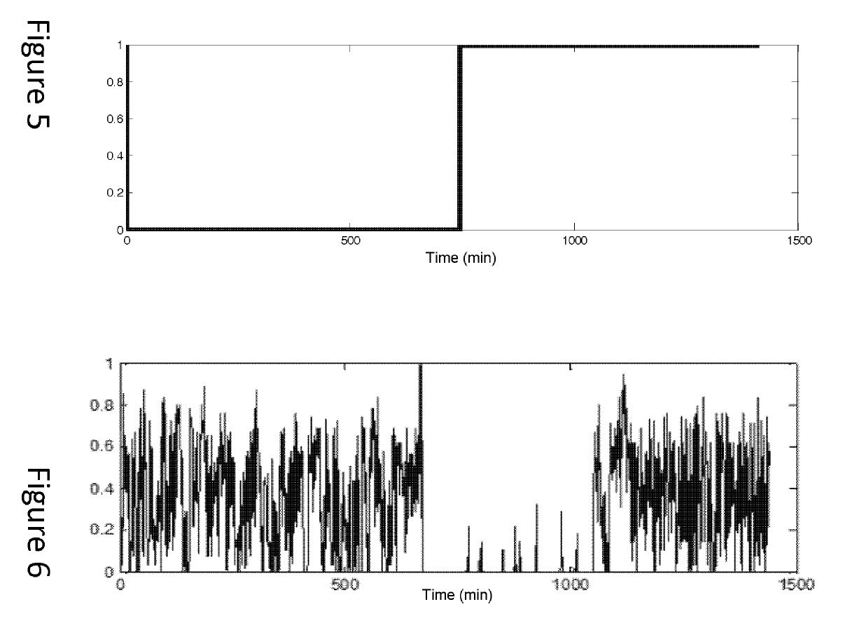

In order to do this, on the whole of the positionX signal, the processor:
determines and records in the memory, the minimum median by carrying out the following sub-steps:
   sorting the values in an increasing order,
   suppressing the value repetitions, in order to obtain a unique data set, and
   calculating the minimum median over the first X (31) values of the unique data set,
determines and records in the memory, the maximum median by carrying out the following sub-steps:
   sorting the values in a decreasing order,
   suppressing the value repetitions, in order to obtain a unique data set, and
   calculating the maximum median over the first X (31) values of the unique data set,
determines and records in the memory, the threshold median corresponding to the average of the minimum and maximum medians,
transforms and records, in the memory, the “positionX” data signal by replacing with 0 the values strictly less than the threshold median, and with 1 the values greater than or equal to the threshold median in order to obtain a positionX_binary data signal (FIG. 4),
smooth out the values recorded in the memory of the positionX_binary data signal, when a set of data delimited by non-identical values corresponds to a time period of less than 1 h30, the values of said data set are then reversed, from 0 to 1 and from1 to 0 (FIG. 5).
   In other words, if the number of binary values contained in the set is less than 90, the binary values of said set are then reversed.

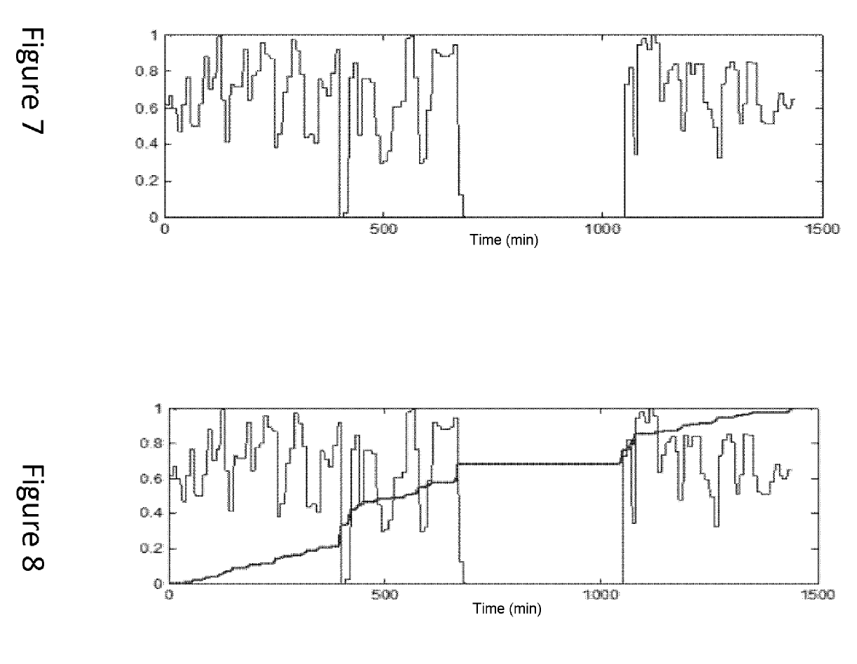

For the transformation into binary values of the “ZCM” values, the processor over the whole of the “ZCM” signal, carries out the following operations:
normalization of the “ZCM” values by dividing each “ZCM” value by the maximum value of the signal in order to obtain a new set of data called ZCM_time for which the values vary between 0 and 1 (FIG. 6),
application to the “ZCM_time” data set, of a variance filter over 10 points.
   Thus, in “ZCM-time” for each point for which the time index i will be modified, the variance filter will range from i to i+9 (FIG. 7).

For example:

ZCM_time[0]=CalculationVariance(ZCM_time [0], ZCM_time[1], . . . ,ZCM_time [9])

ZCM_time[1]=CalculationVariance(ZCM_time [1], ZCM_time[2], . . . ,ZCM_time [10])

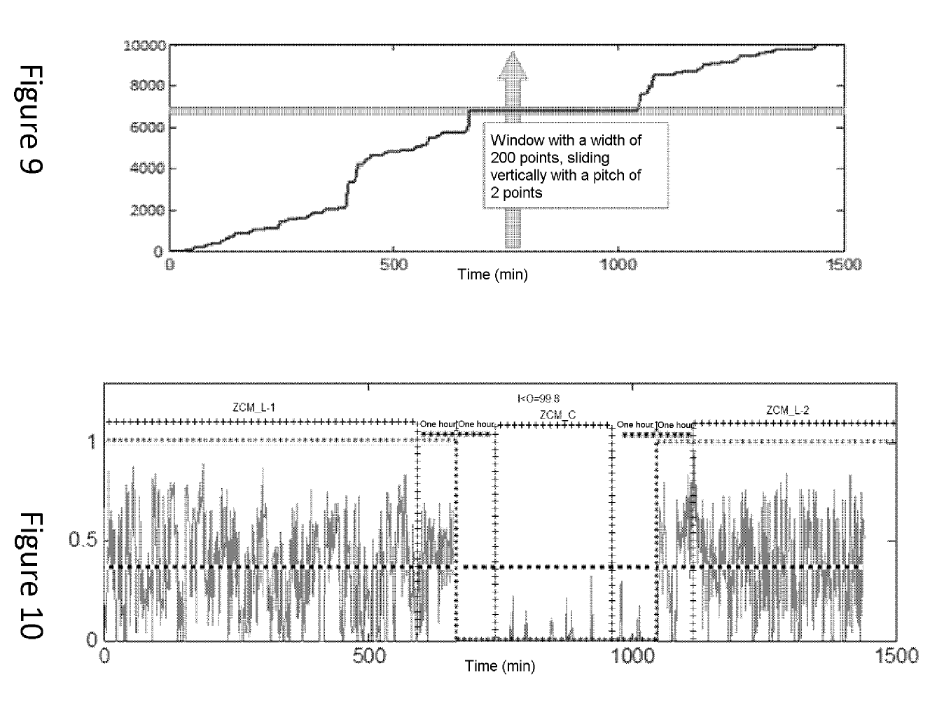

ZCM_time[2]=CalculationVariance(ZCM_time [2], ZCM_time[3], . . . ,ZCM_time [11])
a calculation of the accumulated quadratic average RMS from the ZCM_time signal, i.e. each RMS point (of time index i) is the sum of the preceding RMS point (with a time index i−1) with the result of the RMS over 2 ZCM_time points (FIG. 8).

For example:

RMS[0]=Calculation_RMS(ZCM_time[0], ZCM_time[1])

RMS[1]=Calculation_RMS(ZCM_time[1], ZCM_time[2])+RMS[0]

RMS[2]=Calculation_RMS(ZCM_time[2], ZCM_time[3])+RMS[1]

Also the processor, on the whole of the “ZCM” signal, carries out the following operations:
seeking a plateau on the data of the accumulated quadratic average by carrying out the following sub-steps:
   a multiplication of each value of the quadratic average by a factor of 10⁴ and calculation of the rounded to the nearest integer
   reading for example with a pitch of 2 units a corresponding window, for example with 200 units. In other words, the window gives the possibility of reading up to 200 values of the quadratic average at the same time and is displaced from two to two. I.e. the width of the window is 200 units, and its length is of the size of the signal. (FIG. 11)
   determination of the existence of a plateau when the window comprises at least 180 values of the quadratic average, the program executed by the processor then retains in memory the time index of the first and of the last point of the plateau.
transforms and records in the memory, the “ZCM” data signal by replacing the values located between the time indexes of the plateau with 1 and the other ones with 0 in order to obtain a “ZCM_binary” data signal (FIG. 9).

Following these binary transformation steps, the server compares the binary values of the “ZCM” and “positionX” signals and identifies at least one rest state of the individual, represented by a set of at least 180 binary values equals to 1.

Finally the server calculates the dichotomy index and proceeds with its display on the display means such as a screen, by applying the following equation:

$$I < 0 = ( 1 - \frac{{NB}_{C}}{{NB}_{L}} ) \times 100$$

with:
NB_{C}, the number of values of the first “ZCM” signal representative of the activity of the individual versus time, located in the lying-down state identified during step E, which are greater than the median of the values of the first “ZCM” signal representative of the activity of the individual versus time located outside the lying-down state identified during the preceding step,
NB_{L}, the number of values of the first ZCM signal representative of the activity of the individual, located outside the lying-down state identified during the preceding step.

To do this, the server, by executing the program on its processor, carries out the following sub-steps:
Identification of a set of “ZCM” values stored in memory under the name of “ZCM_C” and the points of which are contained in the plateau,
Identification of a set of “ZCM” values stored in memory under the name of “ZCM_L”, the points of which are on either side of the plateau,
Calculation of the median, “Med_L”, corresponding to the ZCM_L values.
Counting in “ZCM_C”, of the number of points, NB_C, above the median “Med_L” and in “ZCM_L”, of the number of points, NB_L.
Resolution of the equation of the dichotomy index.

In order to improve accuracy, in a preferred embodiment, the processor does not take into account for calculating the dichotomy index, the data corresponding to a time period corresponding to one hour before and one hour after the beginning of the plateau and one hour before and one hour after the end of the plateau. In other words, ZCM_C corresponds to the set of the ZCM values for which the points are contained in the plateau except for the first and last hour of the plateau, and ZCM_L corresponds to the set of ZCM values for which the points are on either side of the plateau except for one hour before the plateau and one hour just after the plateau.

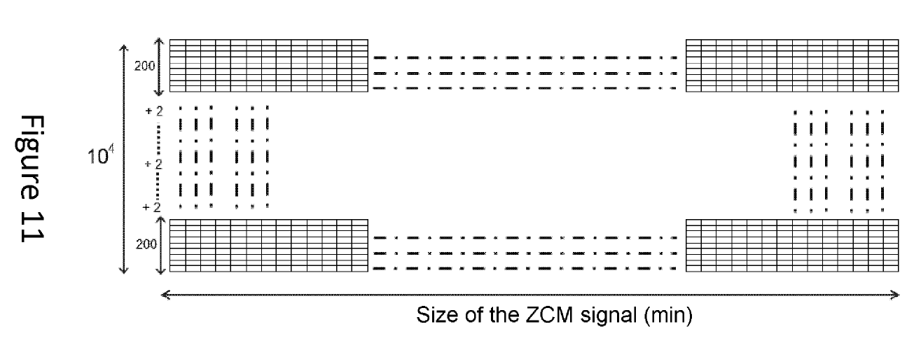

Thus, in this particular embodiment, for calculating the dichotomy index, the latter is only calculated over a period of 20 hours per slice of 24 hours (FIG. 10).

According to another embodiment, if this is required in order to calculate the medians, a set of 16 values identical with the last value of the “ZCM” or “positionX” signal may be added at the end of the signals in order to obtain a sufficient number of values for carrying out the calculation of the medians.

Moreover, when the dichotomy index is greater than 97%, this means that the individual is well asleep and that his/her circadian rhythm is not perturbed.

This result represents a poor prognostic factor in terms of survival in hospitalized individuals, in particular for cancers notably when the dichotomy index is less than 97%.

Obtaining this result gives the possibility of knowing the circadian rhythm of an individual affected with cancer, which allows optimization of the time-modulated administration of the anticancer drugs thereby allowing improvement in tolerance and of the efficiency of the relevant anticancer drugs.

Such a method according to the invention gives the possibility of giving support to a physician with view to elaborating an optimum time-therapeutic scheme for each individual.

In addition, all the variables described above can all be dynamic variables (for example like the 24 hours period described above), which are defined on the basis of measured data and/or statistical estimates. Therefore, a a corrective factor can be added to or subtracted from them. Such corrective factor allows to take the individual variations between the patients into account for a given variable, for example as described herein.

The description of the present invention and the figures related thereto are not provided for limiting the scope of the invention but simply illustrate selected embodiments. One skilled in the art will understand that the technical features of a given embodiment according to a particularity, may in fact be combined with features of another embodiment according to another particularity unless the opposite is explicitly mentioned or if it is obvious that these features are incompatible. Further, the technical features described in a given embodiment according to a particularity, may be isolated from the other features of this embodiment unless the opposite is explicitly mentioned.

It should be obvious for those skilled in the art that the present invention allows embodiments under many other specific forms without departing from the field defined by the scope of the appended claims. They should be considered as an illustration and the invention should not be limited to the details given above.

1. A method for automatically determining the dichotomy index I<O of an individual, from at least data from a system comprising:
a portable module including:
i. a temperature sensor generating a data signal representative of the body temperature of the individual,
ii. an accelerometer simultaneously generating two data signals, a first “ZCM” signal, representative of the activity of the individual over time and a second “positionX” signal representative of the tilt of the individual with respect to the ascending vertical over time,
computing resources including at least one processor and at least one memory, and configured for receiving the data signals from the portable sensor, and for carrying out said method,
wherein the method comprises at least the following steps performed by said processor:
A. receiving and recording in the memory of the various data of the temperature, ZCM and positionX signals of the portable module,
B. suppressing from the memory of the non-utilizable data from the module when the latter is not worn by the individual,
C. identifying and resetting to zero by the processor of the aberrant data from the ZCM and positionX signals of the accelerometer and of their recordings in the memory,
D. transforming the values of the positionX signal of the accelerometer into binary values (0 or 1) corresponding to an alert state (0) or lying-down state (1) of the individual and their recordings in the memory,
E. transforming the values of the ZCM signal of the accelerometer into binary values corresponding to an alert or lying-down state of the individual and their recordings in the memory,
F. comparing the binary values of the ZCM and positionX signals of the accelerometer and identifying at least one lying-down state of the individual constituted by an uninterrupted succession of a binary value identical for the signals of the accelerometer over a time period greater than or equal to 180 minutes,
G. calculating the dichotomy index and its display on the display means, by following the following equation:
$$I < 0 = ( 1 - \begin{matrix}NBc \\ NBL\end{matrix} ) \times 100$$
with:
N Be, the number of values of the first ZCM signal representative of the activity of the individual versus time, located in the lying-down state identified during step E, which are greater than the median of the values of the first ZCM signal representative of the activity of the individual versus time located outside the lying- down state identified during step E,
NBL, the number of values of the first signal relative to the activity (ZCM) of the individual, located outside the lying-down state identified during step E.

2. The method for automatically determining the dichotomy index I<O of an individual according to claim 1, wherein the computing resources are integrated in the portable module for carrying out said method.

3. The method for automatically determining the dichotomy index I<O of an individual according to claim 1, wherein the computing resources are integrated in a computer server distinct from the portable module.

4. The method for automatically determining the dichotomy index I<O of an individual according to claim 3, wherein the computer server comprises display means for displaying the results of the computation.

5. The method for automatically determining the dichotomy index I<O of an individual according to claim 1, wherein the data signals comprise values taken at a regular interval over a time period of 24 hours.

6. The method for automatically determining the dichotomy index I<O of an individual according claim 1, wherein when the number of values of the data signals for the temperature sensor and the accelerometer are different, the processor carries out a normalization step on the data recorded in the memory prior to step B, by using a cubic spline polynomial interpolation so that each value from the temperature signal is assigned to a value of the positionX and ZCM signals, and said values are recorded in the memory of the system.

7. The method for automatically determining the dichotomy index I<O of an individual according to claim 1, wherein, during step B, the processor suppresses from the memory all the values of temperature data strictly less than 30° C. and the values of the positionX and ZCM signals which are associated in time with the temperature value strictly less than 30° C.

8. The method for automatically determining the dichotomy index I<O of an individual according to claim 5, wherein, during step C, the processor independently applies on the whole of the positionX and ZCM data the following sub-steps:
a) Selecting with the processor in the memory a first block of consecutive values,
b) Sorting in an increasing order the values of the block,
c) Suppressing value repetitions of the block, in order to obtain a unique data set,
d) Calculating the median of the unique data set,
e) Resetting to zero in the memory the values of the positionX and ZCM data signals when their value is greater than the median,
f) Repeating steps a) to e) on the next block of consecutive values.

9. The method for automatically determining the dichotomy index I<O of an individual according to claim 6, wherein:
the blocks of values are constituted by 31 values corresponding to a time period of 31 minutes of the signals,
the median is calculated over the first 10 data of the unique data set,
the reset to zero is accomplished by sub-sets of 5 data corresponding to 5 consecutive minutes, if there are less than 2 values greater than the median, then the processor resets to zero in the memory the corresponding sub-set.

10. The method for automatically determining the dichotomy index I<O of an individual according to claim 7, wherein, during step D, on the whole of the positionX signal, the processor:
determines and records in the memory, the minimum median by carrying out the following sub-steps:
sorting the values in an increasing order,
suppressing the value repetitions, in order to obtain a unique data set, and
calculating the minimum median over the first 31 values of the unique data set,
determines and records in the memory, the maximum median by carrying out the following sub-steps:
sorting the values in a decreasing order,
suppressing the value repetitions, in order to obtain a unique data set, and
calculating the maximum median over the first 31 values of the unique data set,
determines and records in the memory, the threshold median corresponding to the average of the minimum and maximum medians
transforms and records, in the memory, the signal of positionX data by replacing with 0 the values strictly less than the threshold median, and with 1 the values greater than or equal to the latter in order to obtain a positionX_binary data signal,
smoothes out the values recorded in the memory of the positionX_binary data signal, by the processor when a set of data delimited by non-identical values corresponds to a time period of less than 1 h30, the values of said data set are then reversed, from 0 to 1 or from 1 to 0.

11. The method for automatically determining the dichotomy index I<O of an individual according to claim 8, wherein, during step E, on the whole of the ZCM signal, the processor
normalizes the ZCM values by dividing each ZCM value by the maximum value of the signal in order to obtain a new ZCM_time signal, the values of which vary between 0 and 1,
applies to the ZCM_time signal a variance filter over 10 points and records the signal in the memory,
calculates the accumulated quadratic average from the ZCM_time signal,
searches for a plateau on the data of the accumulated quadratic average by carrying out with the processor the following sub-steps:
multiplication of each value of the quadratic average by a factor of 10⁴ and calculating the rounded to the nearest integer,
reading with a pitch of 2 units a window corresponding to 200 units,
determining the number of points in each window, if the number of found points corresponds to a time period greater than 180 min, the processor increments a “plateau” counter and retains in memory the time index of the first and of the last point of the plateau,
transforming and recording in the memory, the ZCM data signal by replacing the values located between the time indexes of the plateau withl and the other ones with 0 in order to obtain a ZCM_binary data signal.

12. The method for automatically determining the dichotomy index I<O of an individual according to claim 9, wherein, during step F, the processor carries out a comparison of the ZCM_binary and positionX_binary data signals in order to determine the time indexes of the plateau for which the initial and final data values correspond to 1, the lying-down state.

13. The method for automatically determining the dichotomy index I<O of an individual according to claim 10, wherein, during step G, the processor does not take into account for calculating the dichotomy index, the data corresponding to a time period of one hour before and of one hour after the beginning of the plateau and one hour before and one hour after the end of the plateau.
